(19) United States
(12) Patent Application Publication
(10) Pub. No.: US 2011/0017217 A1
(43) Pub. Date: Jan. 27, 2011

(54) MEDICAL APPARATUSES INCORPORATING DYES

(75) Inventors: Ronald Wood, Rochester, NY (US); Jay E. Reeder, Rochester, NY (US)

Correspondence Address: BLANK ROME LLP, WATERGATE, 600 NEW HAMPSHIRE AVENUE, N.W., WASHINGTON, DC, 20037, US

(73) Assignee: University Of Rochester, Rochester, NY (US)

(21) Appl. No.: 12/672,249

(22) PCT Filed: Aug. 6, 2008
(86) PCT No.: PCT/US08/72371

§ 371 Date: Aug. 12, 2010

Related U.S. Application Data
(60) Provisional application No. 60/954,141, filed on Aug. 6, 2007.

(51) Int. Cl. A61M 16/00

(52) U.S. Cl. 128/207.14; 604/23

(57) ABSTRACT

The invention provided medical apparatuses incorporating dyes to facilitate proper positioning in patients, and methods for their use. The apparatuses can be, for example, tubes, catheters, or needles. In some embodiments, the dye is a near infrared fluorescent dye.

### CROSS-REFERENCES TO RELATED APPLICATIONS

This application claims priority to U.S. Provisional Application No. 60/954,141, filed Aug. 6, 2007, the contents of which are incorporated herein.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK.

Not Applicable

### FIELD OF THE INVENTION

This invention relates generally to medical devices and apparatuses designed to facilitate their proper positioning in patients.

### BACKGROUND OF THE INVENTION

Intubation is the process by which a tube is introduced into the body. For example, nasogastric tubes are inserted through the nose, down the esophagus, into the stomach. Most commonly, the procedure is used to facilitate introducing food into a patient, or to administer drugs, or for other purposes where a conduit is needed for passage of gas or liquids in either direction. Nasogastric tubes are typically positioned by measuring the length from the tip of the nose to the xiphoid process to determine the length of tubing necessary to reach the stomach. Care must be taken not to introduce the tube into the lung. According to a patient safety alert issued by the National Health Service of the United Kingdom on Aug. 18, 2005, studies had shown that many commonly used testing methods to check the placement of nasogastric tubes in adults and neonates could be inaccurate. The Alert cautioned that staff should not rely on tests such as listening (auscultation), the absence of apparent respiratory distress, monitoring of bubbling at the end of the tube, or the appearance of feeding tube aspirate as methods for indicating correct tube placement or misplacement. The Alert further indicated that radiography should not be used routinely in neonates but could be used if the infant was being subjected to radiography for another purpose. Finally, the Alert stated that neonatal units should switch to pH indicator strips or paper since blue litmus paper was not sufficiently sensitive to differentiate between lung and gastric secretions. U.S. Pat. No. 6,984,224 also notes the danger of damage to the vocal cords if the nasogastric tube is misdirected into the trachea instead of the esophagus.

The most common intubation is endotracheal intubation, in which a tube is introduced through the mouth or nose and advanced through the larynx into the trachea and finally into the carina (the point at which the trachea bifurcates into the left and right main bronchi) to maintain an open airway or to permit mechanical ventilation of the patient. The tube is referred to as an endotracheal tube (“ET tube” or “ETT”). As described in U.S. Pat. No. 6,889,693, the oral insertion of an endotracheal tube typically occurs while the patient lies on the back and the neck is slightly extended with the nose in a sniffing position. A caregiver, using his or her left hand to hold a laryngoscope, inserts the laryngoscope into the right corner of the mouth and advances its tip to the depth of the esophagus (swallowing tube into the stomach). The caregiver uses the laryngoscope to displace the tongue to the left side of the mouth, out of the way and providing a line-of-sight into the deepest portion of the patient's throat. This line-of-sight extends into the patient's esophagus, which is a large, wide, funnel-shaped structure. The laryngoscope is withdrawn from the esophagus until the patient's epiglottis and vocal cords (which define an entrance into the patient's trachea) come into view. The epiglottis and the vocal cords lie in front of and above the esophagus. The caregiver then inserts the distal end of the endotracheal tube through the vocal cord opening and into the trachea. After the endotracheal tube is properly positioned in the patient's trachea, the tube is typically secured to the patient's lip (e.g., with tape or a device).

The caregiver cannot see the distal end of an endotracheal tube after it has been inserted through the vocal cords. Consequently, it is difficult to determine the ultimate depth of insertion. Positioning of the ET tube, however, is critical. If the tube is placed into the esophagus instead of the trachea, the tube will not introduce air into the lungs and the patient's life may be placed at risk. As noted in Goodman, L. et al., Am J Roentgenology, 127(3):433-434 (1976), if the tube is placed too close to the vocal cords, there is a risk of extubation, aspiration pneumonia, or laryngeal spasm. If the tube is inserted too far, the tip will extend beyond the carina, typically into the right mainstem bronchus, where it can cause tension pneumothorax and decreased survival. In adult patients, it is usually considered ideal if the tip of the ET tube is about 2 to 6 cm above the carina. This can be determined by radiography to visualize the position of the tip of the tube against the vertebrae, with positioning between T3 and T4 being considered safe. ET tubes often contain a radio-opaque marker at or near the tip to facilitate determining the position of the tip in relation to anatomical structures during radiography, and also typically contain markings every centimeter to facilitate determining the depth to which the tube has been inserted.

Positioning problems are particularly acute in neonates and infants. Premature infants in particular have short tracheas and small carinae, and a number of methods have been devised to estimate the correct length of endotracheal tube to use in such patients, such as correlating the length of the tube to the patient's crown-heel length or crown-rump length. See, e.g., Rotschild et al., Arch Ped Adolescent Med, 145:1007-12 (1991).

Over time, the need for better methods of positioning ET tubes has led to a number of attempted solutions. These include placing indicator devices within the airway to make an audible sound as air moves over the device (U.S. Pat. No. 5,620,004), devices for measuring the oxygen content of inhaled and expired air (U.S. Pat. No. 6,149,603), colorimetric indicators of the carbon dioxide content of the expired air (U.S. Pat. No. 4,879,999), a “marking insert” to be inserted inside the ET tube to permit detecting relative movement (U.S. Pat. No. 4,690,138), ET tubes with a plurality of visually distinct regions differentiated by color (U.S. Pat. No. 6,889,693), and a vibration mechanism coupled to the ET tube to facilitate ultrasonic imaging of the tube (U.S. Published Patent Application 20060081255). The Aug. 18, 2005 patient safety alert of the British National Health Service noted that tubes with radio-opaque markings could be used for neonates to permit accurate determination of depth, length, and tube position, but that radiography should not be used unless the infant was being x-rayed for another reason.

One solution has been a “light wand,” which has a light source at the distal tip that is inserted within the ET tube and is advanced along a curved guide until the light source is externally observable through the tracheal wall (U.S. Pat. No. 6,860,264). A “light wand” approach is embodied in the TrachLight (Laerdal Medical Corp., Armonk, N.Y.), a device having a light at the end for use in visualizing whether an ET tube is inserted down the trachea or down the esophagus. According to the manufacturer, if the ET tube is put down the trachea, the light can be distinctly seen through the tracheal wall, whereas if the ET tube is put down the esophagus, which is more posterior, the glow is more diffuse.

Despite these attempts to provide methods for determining the placement of endotracheal tubes and other devices placed in the body, it would be desirable to have additional means to readily determine the placement of tubes such as ET and nasogastric tubes, as well as other medical tubes and devices placed in the body. The present invention fills these and other needs.

### SUMMARY OF THE INVENTION

In a first group of embodiments, the invention provides medical apparatuses having an exterior surface and including a proximal end and a distal end, which distal end comprises one or more positions marked by a dye. In some embodiments, the apparatus is a tube or catheter. In some embodiments, the tube or catheter is a nasogastric tube. In some embodiments, the tube or catheter is a endotracheal tube. In some embodiments, the medical apparatus is a catheter. In some embodiments, the apparatus is a needle. In some embodiments, the apparatus is a stent not coated with a restenosis-reducing drug. In some embodiments, the dye is a fluorescent dye. In some embodiments, the dye is a near infrared dye. In some embodiments, the dye is on said exterior surface. In some embodiments, the medical apparatus is a tube or catheter and said dye is embedded in said tube or catheter. In some embodiments, a portion of the apparatus permits transmission of excitation and emission wavelengths of the fluorescent dye and the dye is affixed to an interior surface of said portion of said apparatus. In some embodiments, the apparatus is an endotracheal tube adapted for use with an infant or a premature infant. In some embodiments, the medical apparatus is a tube having a tubular member with a length less than about 20 centimeters. In some embodiments, the medical apparatus is an endotracheal tube further comprising a safety marking of fluorescent dye closer to the distal end than the proximal end, wherein the safety marking is adapted for alignment adjacent to a patient's vocal cords. In some embodiments, the medical apparatus is a nasogastric tube adapted for use with an infant or a premature infant. In some embodiments, the nasogastric tube has a tubular member with a length less than about 20 centimeters. In some embodiments, the nasogastric tube further comprises a safety marking of dye closer to the distal end than the proximal end, wherein the safety marking is adapted for alignment adjacent to a patient's vocal cords. In some embodiments, the dye is a fluorescent dye. In some embodiments, the tube or catheter has a line of fluorescent dye extending from said distal to within at least 10 cm of said proximal end. In some embodiments, the medical apparatus comprises a tubular member having said dye disposed throughout said member. In some embodiments, the dye is a fluorescent dye having an excitation frequency and said medical apparatus further comprises a source of light of said excitation frequency (a) integral to the medical apparatus or (b) that can be introduced into said tube or catheter and then withdrawn.

In a further group of embodiments, the invention provides systems comprising (a) a medical apparatus comprising an exterior surface and including a proximal end and a distal end, which exterior surface comprises one or more positions marked by a fluorescent dye, and (b) a visualization apparatus for providing excitation light to said dye and for visualizing fluorescence emitted by said dye in response to said excitation light. In some embodiments, the dye is a near infrared dye and said visualization apparatus comprises a laser emitting near infrared light. In some embodiments, the apparatus comprises a tubular member having an exterior surface, a proximal end and a distal end. In some embodiments, the tubular member has a line of said dye on said exterior surface extending from said distal end to within at least 10 centimeters of said proximal end. In some embodiments, the tubular member has said dye disposed throughout said member.

In yet a further group of embodiments, the invention provides methods of inserting a tube or catheter in a patient, comprising: (a) providing a tube or catheter comprising a tubular member including a distal end and a proximal end, and at least one position on said distal end marked with a dye, (b) inserting the distal end of the tube or catheter into the patient; and (c) aligning said position marked by said dye with an anatomical structure of the patient. In some embodiments, the tube or catheter is an endotracheal tube. In some embodiments, the tube or catheter is an endotracheal tube and the method further comprises: (d) securing the endotracheal tube to the patient using tape after step (c). In some embodiments, the tube or catheter is a nasogastric tube. In some embodiments, the tube or catheter is a nasogastric tube and the method further comprises: (d) securing the nasogastric tube to the patient using tape after step (c). In some embodiments, the dye is a fluorescent dye. In some embodiments, the aligning of said position marked by said dye is by photoacoustic imaging. In some embodiments, the dye is within 3 centimeters of a surface of said patient.

In yet a further group of embodiments, the invention provides systems, comprising (a) a medical apparatus comprising an exterior surface and including a proximal end and a distal end, which exterior surface comprises one or more positions marked by a dye, (b) a visualization apparatus comprising a laser emitting light capable of generating an ultrasonic signal from said dye, and (c) an ultrasonic sensor and imaging device for visualizing ultrasound emitted by said dye in response to said laser light. In some embodiments, the dye is a near infrared dye. In some embodiments, the medical apparatus comprises a tubular member having an exterior surface, a proximal end and a distal end. In some embodiments, the tubular member has a line of said dye on said exterior surface extending from said distal end to within at least 10 centimeters of said proximal end. In some embodiments, the tubular member has said dye disposed throughout said member.

### DETAILED DESCRIPTION

### Introduction

As noted in the Background, the incorrect placement of medical apparatuses, such as tubes and catheters, in patients can cause discomfort, complications, or even death. The present invention provides methods for positioning apparatuses and instruments in patients that overcome the problems experienced with previous approaches. The apparatuses and methods of the invention permit visualizing the position of medical instrumentation and devices placed in patients, facilitating correct placement or rapid correction of incorrect placement.

In the inventive medical instrumentation and devices, a dye is placed on (or, as explained below, inside) the instrumentation or device so that the instrumentation can be visualized in a patient upon illumination of the instrumentation or device. In some embodiments, the dye is a fluorescent dye which, in preferred embodiments, is visualized by illuminating the dye with light of an appropriate frequency. In some alternative embodiments, however, the dye is a fluorescent or non-fluorescent dye that, is imaged by photoacoustic imaging, in which ultrasound is generated by short pulses of laser light and imaged using an ultrasound imaging apparatus.

Visualization of a variety of medical devices and apparatuses can be facilitated by the present invention. In some embodiments, the device or apparatus is a tube or a catheter, to assist in visualizing the tubes or catheters and thereby reduce the problems of placement discussed in the background section. In some embodiments, the device or apparatus is a needle, such as a biopsy needle, needles attached to IV or peripheral venous catheters, or needles for aspiration. For example, it can be difficult to determine whether an IV needle is properly inserted into a vein. Visualization of the needle tip, particularly in combination with commercially available devices for visualizing blood flow, can assist in determining whether the needle is properly inserted. In some embodiments, the device or apparatus is a cannula. In some embodiments, the device or apparatus is an implanted device for electrical stimulation or recording. In some embodiments, the device or apparatus is an implanted drug delivery system, including tubes and pumps designed for the slow release of a therapeutic agent.

In some embodiments, the device or apparatus is a ureteral or urinary stent or a transnasal lacrimal stent. Placement of stents in ureters is currently confirmed by use of a C-arm X-ray device. This not only requires an operator to bring in and position the X-ray device, but all personnel in the operating room must wear lead gowns. In contrast, a stent with embedded or surface dye can be visualized, depending on the dye used, by a fluorescent light source or by photoacoustic imaging source and an ultrasound imager, either of which can be located on a relatively easy-to-maneuver cart or integrated into a handheld device and which requires no special safety precautions other than not looking directly at the laser source if the excitation light is a laser.

The invention can also be used in connection with visualizing transnasal lacrimal stents, such as monocanalicular lacrimal stents. In one group of embodiments, the devices may be designed to permit a slow release of fluorescent material. This permits visualization of the flow of material away from the site of device implantation, for example, through a duct lumen, through the vasculature, or through the lymphatic system. In a further group of embodiments, the slow release of fluorescent materials from drug delivery devices can document the continuity of delivery without the need to determine blood levels of the agent being released from the device. In preferred embodiments, if the device or apparatus is a stent, the stent is not a coronary artery stent or a stent eluting drugs intended to reduce restenosis of the stent. In more preferred embodiments, if the device or apparatus is a stent, the device is a ureteral or urinary stent or a transnasal lacrimal stent.

In preferred embodiments, the tubes, catheters, and other medical apparatuses of the invention are marked with a dye so that the dye can be visualized by illumination of an appropriate wavelength or by photoacoustic imaging. Preferably, the dye is applied in a manner that affixes it permanently to the surface of the tube, catheter or other apparatus. Dye on the exterior surface of the tube, catheter or apparatus will be in contact with body fluids. For those devices in which the practitioner desires to observe the flow of dye from the device, the dye should be non-toxic in any concentration that can become free from the device or apparatus during its placement in the patient. For most embodiments, in which the dye is intended to permit visualization, but not to separate from the device in any appreciable amount, it is preferable that any dye on the exterior surface be in an insoluble form or be non-toxic in any concentration that can dissolve or otherwise become free from the device or apparatus during its placement in the patient.

In some embodiments, the dye is incorporated as a component of a pigment mixture used in current manufacturing processes that currently place lines, rings, dots or characters on the device, or is placed on the device or apparatus by such a process. The dye can also be placed on the device or apparatus and subsequently coated. Many devices are manufactured from multiple separate components and are bound together in an assembly with adhesives or polymerizing agents or by the application of heat to expand or contract thermoplastic materials (such as polyolefin, fluoropolymers and fluoroelastomers, polyvinylchloride, silicone, and similar materials) or to activate thermoplastic adhesives. Dyes may be incorporated in any of these layers or components provided that, in the case of fluorescent dyes, the overlying layers do not block the excitation and emission wavelengths to the degree that fluorescence from the dye cannot be detected through the skin at the depth to which the device or apparatus will normally be used.

The exterior surface of the tube, catheter or other apparatus is particularly convenient for placement of the dye, but it is not the only place the dye can be positioned. The devices or apparatuses can have the dye embedded within them or even (in the case of apparatuses, such as tubes, which have an interior bore defining an interior surface) on an interior surface of the apparatus. For example, the dye can be embedded in one or more positions in the plastic (or other transparent or translucent material) between the interior and exterior surfaces or throughout the entirety of the material. Conveniently, the dye is introduced into the mix of resin or other ingredients before the mix is polymerized to form the plastic, so that the dye is dispersed throughout the device or apparatus into which the plastic is formed. It is expected that persons of skill in manufacturing medical apparatuses such as tubes and catheters are familiar with methods and processes for adding ingredients to them.

In one set of embodiments, the apparatuses are tubes and catheters, with endotracheal and nasogastric tubes being particularly preferred embodiments. Tubes and catheters used in medical applications are typically made of a transparent or translucent plastic acceptable for medical use. Endotracheal and nasogastric tubes, for example, are currently commonly made of polyvinyl chloride, or another medical grade plastic, with a plasticizer added.

Tubes are, of course, tubular structures with a hollow center defined by the surrounded material. As described in the Background, tubes such as endotracheal tubes are inserted down the throat into the lungs. Thus, the tube has a portion which is proximal to the caregiver who is inserting the tube and a distal end, which is the one inserted into the patient's lung, with the tube extending from the proximal end past anatomical structures of concern to the distal end. Similarly, catheters and other devices can be considered to have a proximal end and a distal end, typically the end introduced into the patient, or extending into the patient from the exterior.

In some embodiments, the apparatus has a line of dye applied along at least a portion of its length to facilitate visualization of its placement. For example, with regard to a tube or catheter, typically, the line of the dye commences at or near the tip of the distal end of the tube or catheter, and continues towards the proximal end. Since the proximal end of the tube or catheter will often be visible, it is not necessary for the line of dye to extend all the way to the proximal end. Thus, the line will typically extend from the distal end until the point where the proximal end of the tube or catheter is expected to be clear of anatomical structures of interest. For example, with regard to an endotracheal tube or nasogastric tube, the portion of the tube expected to be above the vocal chords may be left unmarked by dye if desired. Persons of skill will recognize that, in place of a solid line, visualization can be assisted by a line of dots, of squares, or of other markings. In preferred embodiments, the length of the tube or catheter is marked at regular intervals to facilitate calculation of depth or distance. For example, the tube or catheter can be marked by dye at 1 centimeter intervals, at ¼ inch intervals, or at such other interval as is desired by practitioners to facilitate determining proper positioning of the apparatus in question.

While the invention is particularly useful for the placement of tubes and catheters, dye can also be used to mark other medical apparatuses that are implanted near the surface of a patient's body to facilitate confirming their placement and other parameters. Where such an apparatus has a tubular member, for example, the tubular member can be marked with dye to permit visualization of the tubular member.

The devices and apparatuses are generally used as a component of a system that comprises not only the device or apparatus having disposed on or in it the dye, but also a visualization apparatus. For example, in embodiments wherein the visualization is of fluorescent dye, the system includes a device providing excitation light to the dye and a device for visualizing fluorescence emitted by the dye in response to the excitation light. Where the dye is a near infrared dye, the visualization apparatus will preferably be a laser emitting near infrared light, although other light sources of appropriate wavelength for dye excitation may be used if desired. In some embodiments in which the device or apparatus has a bore, such as a nasogastric tube, the apparatus may also include an integral light source or one that touches or surrounds a device made of a clear or translucent material, so that the device itself acts as a light tube to transmit light to the dye. In some embodiments, the light source can be introduced to facilitate positioning and then withdrawn. Since the light is intended to trigger fluorescence from the dye, and it is the fluorescence from the dye that will be visualized to determine the position of the device or apparatus, the light source does not have to be as intense as when used transcutaneously as would a light intended to be seen directly. This may reduce the regulatory burden when a laser is used as the light source by allowing use of a laser of lower classification, and increases the safety factor, as the amount of illumination used can be lessened compared to an external administered light source. Further, in preferred embodiments, the dye is a near infrared dye and the light source emits an excitation light that is in the near infrared and not visible to the unaided eye.

In embodiments in which the visualization is by photoacoustic imaging, the system includes a laser capable of rapid pulsing, preferably in the near infrared, an ultrasound sensor, and an apparatus for imaging ultrasound generated by the laser.

The invention further provides methods for improving the visualization of devices or apparatuses in a patient. For example, the invention provides a method of inserting a tube or catheter into a patient, comprising providing a tube or catheter comprising a tubular member including a distal end and a proximal end, and at least one position on said distal end marked with a fluorescent dye, inserting the distal end of the tube or catheter into the patient; and aligning the position marked by the dye with an anatomical structure of the patient. In some embodiments, the tube is an endotracheal tube or a nasogastric tube. The tube may be taped in place once it is inserted to the desired position.

A study of imaging fluorescence of ICG in the lymphatic system reported that trafficking of ICG could be visualized by near infrared illumination in lymph nodes which were located as deep as 3 cm beneath the tissue surface. Sharma et al., “Quantitative imaging of lymph function,” Am J Physiol Heart Circ Physiol, 292:H3109-H3118 (2007) (first published Feb. 16, 2007; doi:10.1152/ajpheart.01223.2006). Accordingly, in some embodiments, the dye in or on a device or apparatus whose positioned is to be determined is located 3 cm or less beneath the surface of the patient's skin. In some embodiments, the dye in or on a device or apparatus whose positioned is to be determined is located 2.5 cm or less beneath the surface of the patient's skin. In some other embodiments, the dye in or on a device or apparatus whose positioned is to be determined is located 2 cm or less beneath the surface of the patient's skin. In yet other embodiments, the dye in or on a device or apparatus whose positioned is to be determined is located 1.5 cm, 1 cm, 0.5 cm, 0.25 cm, or less beneath the surface of the patient's skin, with each lesser distance below the surface being progressively more preferred. Such distances are appropriate to, for example, visualize the location of a device or apparatus in the trachea of a neonate or infant. Since the near infrared illumination in the Sharma et al. study was able to cause fluorescence of ICG as deep as 3 cm., it is anticipated that at least the depths mentioned above can also be visualized by photoacoustic imaging.

Imaging using fluorescence

In some preferred embodiments, the invention uses fluorescent dyes, which are then visualized by imaging fluorescence of the dye using light of a suitable excitation frequency. Persons of skill are aware that fluorescent dyes have characteristic excitation frequencies and emission frequencies. Dyes fluorescing at various frequencies can be used in the compositions, systems, and methods of the invention. Because near infrared light can penetrate a greater distance into the body than visible light, near infrared fluorescent (“NIRF”) dyes can permit instrumentation or apparatuses within the patient but close to the surface of the skin to be seen, thereby permitting visualization and confirmation of the position of the instrumentation or apparatus. NIRF and other dyes that can be excited and visualized through skin are therefore preferred in the compositions, systems, and methods of the invention, with NIRF dyes being particularly preferred. For convenience of reference, portions of the discussion below pertaining to embodiments using fluorescent dyes visualized by imaging fluorescence of the dye (as opposed visualization of a fluorescent dye by photoacoustic imaging) will focus on NIRF dyes.

Photoacoustic imaging

Photoacoustic imaging is a relatively recent and developed technique that permits imaging body tissues using very short pulses of laser light, typically in the nanosecond range, to generate ultrasound, which is then imaged using a conventional ultrasound probe and imaging apparatus. Typically, for biomedical imaging, the laser is a near infrared laser, since this is an optical window to which body tissues are relatively transparent. It is assumed that the practitioner is generally familiar with the considerable literature on the subject, such as Xu and Wang, “Photoacoustic imaging in biomedicine,” Rev. Sci. Instrum., 77, 041101 (2006); DOI:10.1063/1.2195024; Wang et al., “Imaging of joints with laser-based photoacoustic tomography: An animal study,” Medical Physics, 33(8):2691-2697 (2006) (hereafter, “Wang et al., 2006”); Wang et al., “Noninvasive photoacoustic tomography of human peripheral joints toward diagnosis of inflammatory arthritis, Opt Lett., 32(20):3002-4 (2007) (hereafter, “Wang et al., 2007”); Kolman et al., “In vivo photoacoustic imaging of blood vessels with a pulsed laser diode,” Lasers Med Sci., 21(3):134-9 (2006) (hereafter, “Kolman et al., 2006”); and Bost and Lemor, “Photoacoustic microscopy for high-resolution imaging,” J Acoust Soc Am., 123(5):3370 (2008). Photoacoustic imaging using an intensity modulated, continuous wave laser has also been reported. See, Maslov and Wang, “Photoacoustic imaging of biological tissue with intensity-modulated continuous-wave laser,” J Biomed Opt., 13(2):024006 (2008). The design of laser diodes tunable to different near infrared frequencies is discussed, for example, in Allen and Beart, “Pulsed near-infrared laser diode excitation system for biomedical photoacoustic imaging,” Opt. Lett., 31(23):3462-3464 (2006). Persons of skill are considered capable of choosing suitable laser and dye combinations.

For purposes of the present invention, photoacoustic imaging can be used to facilitate placement of the device or apparatus so long as the practitioner can see the device or apparatus, or can see a contrast between the device or apparatus and the portion of the body in which the device or apparatus is being positioned, or both. Dyes, whether fluorescent or non-fluorescent, can serve as contrast agents, enhancing the ability to visualize the device or apparatus, or the contrast between the device or apparatus, against the surrounding tissue or organs. In some embodiments, the photoacoustic imaging is conducted using a contrast agent, whether or not the contrast agent is classified as a dye. Some contrast agents suitable for use in photoacoustic imaging are disclosed in Guccione, United States Published Patent Application 2008/0181851, and Achilefu et al., United States Published Patent Application 2008/0008655.

Work in imaging body tissues has previously shown that the near infrared fluorescent dye indocyanine green (“ICG”), whose excitation wavelength was close to that of a laser used for photoacoustic imaging, enhanced optical contrast. Ku and Wang, “Deeply penetrating photoacoustic tomography in biological tissues enhanced with an optical contrast agent,” Opt. Lett., 30(5):507-509 (2005). ICG has also been embedded in silicate nanoparticles conjugated to antibodies for targeting of cancer cells for photoacoustic imaging. See, Kim et al., “Indocyanine-green-embedded PEBBLEs as a contrast agent for photoacoustic imaging,” J Biomed Opt., 12(4):044020 (2007). ICG is particularly preferred for use as a dye in the devices, apparatuses, and methods of the invention.

As noted, an ultrasound sensor typically detects the ultrasound generated by the laser pulses. Since the ultrasound signal is produced by the effect of laser photons, the ultrasound sensor can be a receiver, rather than a transducer, which by definition both generates and receives ultrasound. Ultrasound transducers such as those used in normal medical ultrasound examinations, are however, already widely available in combination with systems for processing the received signals into visual images and may be adapted for use in photoacoustic imaging. Methods for imaging ultrasound generated by the laser pulses are known in the art, as evidenced by the imaging of joints and blood vessels reported in Wang et al., 2006,supra, Wang et al., 2007, supra, and Kolman et al., 2006, supra. See also, Wang et al., United States Patent Application 2008/0173093.

Dyes for imaging

Persons of skill will appreciate that a considerable literature is available in the art on the characteristics of different dyes, including, for fluorescent dyes, their excitation wavelength and emission wavelength. This literature is well known, and will not be set forth in detail herein. Both fluorescent and non-fluorescent dyes can be used where photoacoustic imaging is the method chosen for visualizing the device or apparatus, as the imaging will be of ultrasound, while visualization by imaging fluorescence will of course require a fluorescent dye. Since both techniques can use fluorescent dyes, for ease of reference, the discussion below will discuss fluorescent dyes in particular.

A fluorescent dye is imaged by exciting it with a light that has a wavelength appropriate for the particular dye used. Persons of skill are aware that a variety of dyes exist, and that each dye has a known excitation wavelength and a known emission wavelength. Some dyes, for example, fluoresce under ultraviolet (“UV”) illumination while others fluoresce under visible illumination. There is a large literature on the use of fluorescent dyes and probes in biological assays, such as Dewey, T. G., Ed., Biophysical and Biochemical Aspects of Fluorescence Spectroscopy, Plenum Publishing (1991), Guilbault, G. G., Ed., Practical Fluorescence, Second Edition, Marcel Dekker (1990), Lakowicz, J. R., Ed., Topics in Fluorescence Spectroscopy: Techniques (Volume 1, 1991); Principles (Volume 2, 1991); Biochemical Applications (Volume 3, 1992); Probe Design and Chemical Sensing (Volume 4, 1994); Nonlinear and Two-Photon Induced Fluorescence (Volume 5, 1997); Protein Fluorescence (Volume 6, 2000); DNA Technology (Volume 7, 2003); Plenum Publishing, Lakowicz, J. R., Principles of Fluorescence Spectroscopy, Second Edition, Plenum Publishing (1999), and W. T. Mason, ed., Fluorescent and Luminescent Probes for Biological Activity. A Practical Guide to Technology for Quantitative Real-Time Analysis, Academic Press (Second Ed., 1999).

Preferred fluorescent dyes suitable for use in the compositions and methods of the invention are non-toxic dyes which fluoresce when exposed to radiant energy, e.g. light. Preferably, the dyes are near infrared fluorochromes that emit light in the near infra red spectrum, which is within the “optical window” of tissue, where absorption due to endogenous chromophores is low. In some embodiments, the dye is a tricarbocyanine dye, and in particularly preferred embodiments, is indocyanine green (“ICG”). ICG is commercially available from, for example, Akorn, Inc. (Buffalo Grove, Ill.), which sells it under the name IC-GREEN™. In other embodiments, near infrared fluorescent compounds are selected from those developed by Eastman Kodak and/or other manufacturers and available in the market place for other applications requiring invisible printing of identifying marks, barcodes or other graphic or alphanumeric material. Such dyes are achieved, for example, by incorporating metals (e.g. gold), metal salts, organometallic complexes or compounds, quantum dots, nanoparticles or nanotubes. Choice among fluorescent materials may be made on the basis of intensity of the fluorescence; the toxicity of the dye is irrelevant when embedded in a substrate from which release is impossible or so small during the period of use that the risk to the patient may be considered negligible.

In other embodiments the dye is selected from fluorescein isothiocyanate, rhodamine, phycoerythrin, phycocyanin, allophycocyanin, o-phthaldehyde, fluorescamine, Rose Bengal, trypan blue, and fluoro-gold. The dyes may be mixed or combined. In some embodiments, dye analogs may be used. A “dye analog” is a dye that has been chemically modified, but still retains its ability to fluoresce when exposed to radiant energy of an appropriate wavelength. Some dyes, such as ICG, have been used in mammals with low evidence of toxicity and are preferred for embodiments in which the dye may come in direct contact with bodily fluids.

ICG is particularly preferred both because it has low toxicity and because it has been approved by the Food and Drug Administration for several diagnostic purposes in humans. Its absorption (excitation) and emission peaks (805 and 835 nm, respectively), which is within the optical window of tissue. Near infrared light can therefore penetrate tissue from a depth of several millimeters to a few centimeters. ICG is not metabolized in the body and is excreted exclusively by the liver, with a plasma half-life of 3 to 4 minutes. It is not reabsorbed from the intestine and does not undergo enterohepatic recirculation. Commercially available ICG contains iodine and is not recommended for administration to persons with a history of iodine sensitivity. Again, this is not expected to be an issue for uses in which the dye is embedded in the device, such as in a catheter. The recommended dose for ICG video angiography is 0.2 to 0.5 mg/kg; the maximum daily dose should not exceed 5 mg/kg. The amounts used in devices or apparatuses would therefore offer a de minimis risk of toxicity.

For visualizing the tube, catheter, apparatus or instrumentation, by fluorescence (as opposed to the photoacoustic imaging discussed elsewhere herein) the area in which imaging is desired is illuminated with a light of the excitation wavelength or wavelengths suitable for the dye or dyes used. Ambient light may need to be dimmed to permit the detection of fluorescence, depending on the presence of interfering wavelengths (strong incandescent illumination in the case of near IR illumination) or on the characteristics of the fluorescence detection system. If the tube, catheter, apparatus or instrumentation is small, magnification may be needed to facilitate observation. Where the excitation wavelength is outside of the visible range (where, for example, the excitation wavelength is in the ultraviolet or near infrared range), the light source may be designed to permit switching or “toggling” between light for general illumination and the excitation wavelength. This permits the practitioner to note the position of the tube, catheter, apparatus or instrumentation in relation to the rest of the body and surrounding (but non-fluorescent) structures using visible or broad spectrum near IR light and then to determine the position of the tube, catheter, apparatus or instrumentation. Pseudocolor methods may be used to highlight fluorescent areas in a contrasting color superimposed upon a normal color or grey-scale image of the area of interest.

As noted in the preceding section, the dye will typically be on the tube, catheter, apparatus or instrumentation, or embedded in it if the material is transparent or translucent.

Visualization by photoacoustic imaging uses an ultrasound sensor and ultrasound imaging systems adapted for photoacoustic imaging.

Instrumentation for imaging by fluorescence

As discussed in the preceding section, a number of fluorescent dyes are available for use in connection with the compositions, methods and systems of the invention. Persons of skill will recognize that the light source must be selected to provide illumination optimized for the excitation frequency suitable for the particular dye chosen and similarly, that the device for capturing the light emitted by the dye selected must be able to receive light of the emission frequency of the dye selected. For convenience, the following discussion will refer generally to instrumentation optimized for use with an exemplar near infrared dye, indocyanine green (“ICG”). It is expected that, in light of the discussion herein persons of skill are able to adjust the instrumentation as necessary for any particular fluorescent dye.

Conveniently, the device used for visualization of the tube, catheter, or other apparatus or instrumentation in the area of interest comprises both a laser and a camera. For use with ICG, for example, the laser preferably consists of a laser diode providing a maximum of 3 W output at 806 nm. The laser output is decollimated (i.e. optics are used to spread out the laser light from a tight beam) to provide even illumination over a field of view, for example, 7.6 cm by 7.6 cm at a working distance of 30 cm. The unit typically contains a charge-coupled device (“CCD”) video camera sensitive into the near infrared spectrum and, for use with ICG, is equipped with an 815 nm edge filter. An articulated arm, connected to a wheeled base, supports the laser/camera device. This allows the imaging head to be moved into close proximity to the treatment area and for vertical movement of the head to attain the correct focal distance above the area of interest, such as the throat of a patient being intubated. If desired, the imaging head and extension arm that protrudes over the treatment area may be covered with an optically transparent sterile drape. The laser can be activated by means of a computer command or by foot pedal. Laser/camera devices suitable for intra-operative imaging are commercially available. In some preferred embodiments, the laser/camera device is a SPY® Intra-operative Imaging System, a HELIOS® Imaging System, or a SPY® Intra-Operative Imaging System (all by Novadaq Technologies, Inc., Mississauga, Ontario, Canada). In some embodiments, the illumination system can be a handheld device permitting illumination and visualization. For example, PULSION Medical Systems AG (Munich, Germany) offers a Pulsion© IC-VIEW system consists of a digital video camera and an infrared light source and permits real time visualization of ICG fluorescence in the camera. Alternatively, the area of interest can be illuminated with an excitation frequency appropriate for the dye selected from a lamp either fixed or on a boom or other movable source, and the resulting fluorescence detected by a handheld device, such as a digital camera adapted for reception of light of the emission frequency for the dye in question.

For visualizing the apparatus, an 806 nm excitation light causes the dye to fluoresce, emitting light maximally at 830 nm. The emitted light can then be imaged directly or, preferably, is captured using an imaging system. Typically, the capture system is a charge-coupled device (CCD) camera or CMOS (complementary symmetry metal oxide semiconductor) image sensor, which feeds the image to a monitor so that the caregiver can visualize the fluorescence of the dye in the tube, catheter, or other apparatus or instrumentation in the area of interest in real time. Filters limit the light detected to a range appropriate for the selected fluorescence wavelengths. Optionally, the camera is also attached to a computer and the image is saved, which not only permits documentation of the position of, for example, an endotracheal tube in the patient's carina, but also can be used for training nurses, doctors, and other medical staff who may need to introduce instrumentation into patients. Typically, the time required for positioning the device is 2 minutes, while the total time that the tube, catheter, or other apparatus or instrumentation is illuminated with laser light is 30 seconds.

In some embodiments, an instrument having an optical configuration similar to a fluorescence microscope may be used, in which a dichroic mirror is used to split the paths of the illumination (the excitation light). The excitation light reflects off the surface of the dichroic mirror into the objective, while the fluorescence emission passes through the dichroic mirror to the eyepiece or is converted into a signal to be presented on a screen. The instrument may further have an excitation filter or an emission filter, or both, to select the wavelengths appropriate for each function. Conveniently, the filters are interference filters, which block transmission of frequencies out of their bandpass.

### EXAMPLES

The following examples are offered to illustrate, but not to limit the claimed invention.

### Example 1

An endotracheal tube with near infrared (NIR) fluorescent rings positioned at 1 cm intervals on the exterior surface along the length of the tube is used to intubate a patient. Positioning of the tube is verified with a laser-fluorescence imaging device (Novadaq Technologies, Inc., Mississauga, Ontario, Canada) consisting of a NIR laser light source and a NIR-sensitive digital camcorder. For measurements, the unit is positioned 30 to 40 cm from the area of interest. The NIR light emitted by the laser light source induces ICG fluorescence. The fluorescence is visualized by the practitioner, who determines from the position of the fluorescing rings whether the tube is in the desired position. The fluorescence is also recorded by a digital video camera, with optical filtering to block ambient light. Images can be observed on screen in real time (25 or 30 images/sec). The frame rate can be slowed if desired to permit the device or apparatus to be imaged at deeper depths. The images can be reviewed and stored on the digital video camera or transferred to a computer or to storage media for quality control and risk management purposes.

### Example 2

An endotracheal tube with rings of indocyanine green (“ICG”) dye positioned at 1 cm intervals on the exterior surface along the length of the tube is used to intubate a patient. Positioning of the tube is verified using a laser-diode providing nanosecond pulses of laser light at 800 nm positioned 30 to 40 cm from the area of interest. The light emitted by the laser light source induces ultrasound permitting contrast between the ICG and the patient's tissue. An ultrasound sensor and imaging system is used to visualize the ultrasound generated by the laser light emissions. The contrast is visualized by the practitioner, who determines from visualizing the position of the dye rings whether the tube is in the desired position. Images can be observed on screen in real time (25 to 30 images/sec). The images can be reviewed and stored on the digital video camera or transferred to a computer or to storage media for quality control and risk management purposes.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A medical apparatus having an exterior surface and including a proximal end and a distal end, which distal end comprises one or more positions marked by a dye.

2. The medical apparatus of claim 1, wherein said apparatus is a tube or catheter.

3. The medical apparatus of claim 2, wherein said tube or catheter is a nasogastric tube.

4. The medical apparatus of claim 2, wherein said tube or catheter is a endotracheal tube.

5. The medical apparatus of claim 1, wherein said medical apparatus is a catheter.

6. The medical apparatus of claim 1, wherein said apparatus is a needle.

7. The medical apparatus of claim 1, wherein said apparatus is a stent not coated with a restenosis-reducing drug.

8. The medical apparatus of claim 1, wherein said dye is a fluorescent dye.

9. The medical apparatus of claim 1, wherein said dye is a near infrared dye.

10. The medical apparatus of claim 1, wherein said dye is on said exterior surface.

11. The medical apparatus of claim 1, wherein said medical apparatus is a tube or catheter and said dye is embedded in said tube or catheter.

12. The medical apparatus of claim 8, wherein a portion of said apparatus permits transmission of excitation and emission wavelengths of said fluorescent dye and said dye is affixed to an interior surface of said portion of said apparatus.

13. The medical apparatus of claim 4, wherein the medical apparatus is an endotracheal tube adapted for use with an infant or a premature infant.

14. The medical apparatus of claim 4, wherein the medical apparatus is a tube having a tubular member with a length less than about 20 centimeters.

15. The medical apparatus of claim 4, wherein the medical apparatus is an endotracheal tube further comprising a safety marking of fluorescent dye closer to the distal end than the proximal end, wherein the safety marking is adapted for alignment adjacent to a patient's vocal cords.

16. The medical apparatus of claim 3, wherein the medical apparatus is a nasogastric tube adapted for use with an infant or a premature infant.

17. The medical apparatus of claim 16, wherein the nasogastric tube has a tubular member with a length less than about 20 centimeters.

18. The medical apparatus of claim 16, wherein said nasogastric tube further comprises a safety marking of dye closer to the distal end than the proximal end, wherein the safety marking is adapted for alignment adjacent to a patient's vocal cords.

19. The medical apparatus of claim 18, wherein said dye is a fluorescent dye.

20. The medical apparatus of claim 2, wherein said tube or catheter has a line of fluorescent dye extending from said distal to within at least 10 cm of said proximal end.

21. The medical apparatus of claim 1, wherein said medical apparatus comprises a tubular member having said dye disposed throughout said member.

22. The medical apparatus of claim 2, wherein said dye is a fluorescent dye having an excitation frequency and said medical apparatus further comprises a source of light of said excitation frequency (a) integral to the medical apparatus or (b) that can be introduced into said tube or catheter and then withdrawn.

23. A system comprising (a) a medical apparatus comprising an exterior surface and including a proximal end and a distal end, which exterior surface comprises one or more positions marked by a fluorescent dye, and (b) a visualization apparatus for providing excitation light to said dye and for visualizing fluorescence emitted by said dye in response to said excitation light.

24. The system of claim 23, wherein said dye is a near infrared dye and said visualization apparatus comprises a laser emitting near infrared light.

25. The system of claim 23, further wherein said apparatus comprises a tubular member having an exterior surface, a proximal end and a distal end.

26. The system of claim 25, wherein said tubular member has a line of said dye on said exterior surface extending from said distal end to within at least 10 centimeters of said proximal end.

27. The system of claim 25, wherein said tubular member has said dye disposed throughout said member.

28. A method of inserting a tube or catheter in a patient, the method comprising: (a) providing a tube or catheter comprising a tubular member including a distal end and a proximal end, and at least one position on said distal end marked with a dye, (b) inserting the distal end of the tube or catheter into the patient; and (c) aligning said position marked by said dye with an anatomical structure of the patient.

29. The method of claim 26, wherein said tube or catheter is an endotracheal tube.

30. The method of claim 29, further comprising: (d) securing the endotracheal tube to the patient using tape after step (c).

31. The method of claim 28, wherein said tube or catheter is a nasogastric tube.

32. The method of claim 31 further comprising: (d) securing the endotracheal tube to the patient using tape after step (c).

33. The method of claim 28, wherein said dye is a fluorescent dye.

34. The method of claim 28, wherein said aligning of said position marked by said dye is by photoacoustic imaging.

35. The method of claim 28, wherein said dye is within 3 centimeters of a surface of said patient.

36. A system comprising (a) a medical apparatus comprising an exterior surface and including a proximal end and a distal end, which exterior surface comprises one or more positions marked by a dye, (b) a visualization apparatus comprising a laser emitting light capable of generating an ultrasonic signal from said dye, and (c) an ultrasonic sensor and imaging device for visualizing ultrasound emitted by said dye in response to said laser light.

37. The system of claim 36, wherein said dye is a near infrared dye.

38. The system of claim 36, further wherein said medical apparatus comprises a tubular member having an exterior surface, a proximal end and a distal end.

39. The system of claim 38, wherein said tubular member has a line of said dye on said exterior surface extending from said distal end to within at least 10 centimeters of said proximal end.

40. The system of claim 38, wherein said tubular member has said dye disposed throughout said member.
